(19) United States
(12) Patent Application Publication
(10) Pub. No.: US 2026/0080125 A1
(43) Pub. Date: Mar. 19, 2026

(54) METHOD AND SYSTEM FOR GENERATING TARGET MOLECULE

(71) Applicant: Quantiphi, Inc, Marlborough, MA (US)

(72) Inventors: Dagnachew Birru, Marlborough, MA (US); Siddartha Reddy Nareddy, Mumbai (IN); Venkata Sai Prakash Mukkamala, Mumbai (IN); Saisubramaniam Gopalakrishnan, Mumbai (IN); Vishal Vaddina, Toronto (CA)

(73) Assignee: Quantiphi, Inc, Marlborough, MA (US)

(21) Appl. No.: 18/887,245

(22) Filed: Sep. 17, 2024

(51) Int. Cl. G06F 30/27; G16C 20/50; G16C 20/70

(52) U.S. Cl. CPC G06F 30/27; G16C 20/50; G16C 20/70

(57) ABSTRACT

Disclosed is method for generating a target molecule (412, 514, 802), comprising receiving first user input (806) indicative of properties associated with target molecule, and identifying properties (808A-C) associated with targeted molecule and corresponding objectives (810A-B); generating property scores (832A-B) for properties using property predictor algorithm (812); receiving second user input indicative of molecular structure (202, 402, 502, 602, 702, 814) of input molecule (204); generating corresponding target molecules (CTMs) (200, 406, 508, 600, 700, 816); generating embeddings (824) of CTMs; determining aggregate similarity score (828); determining aggregate property score; determining fitness scores (410, 512, 834) of CTMs; determining whether given target molecule amongst CTMs fulfill termination criteria (TC); when it is determined that TC is fulfilled by given target molecule, deeming given target molecule as target molecule to be generated; when it is determined that TC is not fulfilled, updating generated CTMs, iteratively performing steps (v) to (ix).

### FIELD OF TECHNOLOGY

The present disclosure generally relates to drug discovery. Specifically, the present disclosure relates to a method and a system for generating target molecules.

### BACKGROUND

Generally, discovery and development of new drug molecules is a complex and resource-intensive process. Traditional methods of drug discovery often involve extensive laboratory work and costly trials, making the process slow and inefficient. However, with advancement of computational techniques, there is a significant shift towards utilizing machine learning and artificial intelligence to streamline and accelerate the process of drug discovery.

Existing solutions for drug discovery involve use of various machine learning models to predict and optimize molecular properties. In an existing solution the use of variational autoencoders (VAEs) is demonstrated in LIMO (Latent Inceptionism for Targeted Molecule Generation). LIMO employs the VAEs to create a latent space where molecules are represented, followed by a property prediction mechanism using neural networks to optimize molecular properties. However, the aforementioned solution fails to include non-differentiable oracles, limiting its effectiveness in generating the targeted molecules that meet all desired criteria. In another existing solution, a stoned algorithm, utilizes evolutionary algorithms to optimize molecular properties through string manipulations of molecular structures encoded in SELFIES. However, while being effective in exploring molecular space, the solution does not leverage the latent space optimization. In yet another existing solution, such as Junction Tree Variational Autoencoder (JT-VAE) approach, molecular graphs are generated by first creating a tree-structured scaffold and then combining these substructures into a molecule. The solution uses gradient-based optimization to fine-tune molecular properties. Although, the JT-VAE approach is limited by its reliance on gradient-based techniques, which can be less effective when dealing with complex, non-linear property landscapes or non-differentiable objectives.

Furthermore, the existing solutions generally do not incorporate toxicity evaluations, which can result in the generation of molecules that are not suitable for therapeutic use due to potential toxicity issues. Furthermore, the inability to incorporate non-differentiable oracles, the dependence on gradient-based optimization, and the lack of integrated toxicity evaluation constrain the effectiveness and applicability of the existing solutions.

Therefore, in light of the foregoing discussion, there exists a need to overcome the aforementioned drawbacks.

### BRIEF SUMMARY OF THE DISCLOSURE

The present disclosure provides a method and a system for generating a target molecule. The present disclosure seeks to provide a solution to the existing problem of how to simplify and automate a process of generation of a target molecule for drug discovery. The aim of the present disclosure is to provide a solution that overcomes at least partially the problems encountered in the prior art and provide an improved method and system for targeted generation of molecules with genetic algorithm optimization and differential evolution optimization which simplifies and automates the process of generating the target molecule.

In one aspect, the present disclosure provides a method for generating a target molecule. The method comprises receiving a first user input indicative of properties associated with the target molecule, and identifying a plurality of properties associated with the targeted molecule and a plurality of corresponding objectives, therefrom, wherein each property amongst the plurality of properties is associated with a corresponding objective amongst the plurality of corresponding objectives. Moreover, the method comprises generating property scores for the identified plurality of properties using a property predictor algorithm. Furthermore, the method comprises receiving a second user input indicative of a molecular structure of an input molecule. Furthermore, the method comprises generating corresponding target molecules, based on the molecular structure of the input molecule, using a Variational Autoencoder (VAE) module. Furthermore, the method comprises generating embeddings of the corresponding target molecules, using a contrastive pretrained molecule encoder. Furthermore, the method comprises determining an aggregate similarity score based on similarity scores between the embeddings of the corresponding target molecules and embeddings of key relevant information extracted from the first user input. Furthermore, the method comprises determining an aggregate property score based on the identified plurality of objectives and the property scores of the identified plurality of properties. Furthermore, the method comprises determining fitness scores of the corresponding target molecules, based on the aggregate similarity score and the aggregate property score. Furthermore, the method comprises determining whether a given target molecule amongst the corresponding target molecules fulfill a termination criteria and when it is determined that the termination criteria is fulfilled by the given target molecule, deeming the given target molecule as the target molecule to be generated. Furthermore, the method comprises determining whether a given target molecule amongst the corresponding target molecules fulfill a termination criteria and when it is determined that the termination criteria is not fulfilled by the given target molecule, updating the generated corresponding target molecules and iteratively performing steps (v) to (ix).

Beneficially, the embodiments of the present disclosure provide a simplified, efficient and automated method that accurately generate the target molecule with desired properties by utilizing the Natural Language Processing (NLP) and the Named Entity Recognition (NER) algorithms. The use of the disclosed method removes a need for human intervention and simplifies the process of generating the target molecule with desired properties for drug discovery. Moreover, the disclosed method significantly increased a speed of generating the target molecule for the drug discovery. Furthermore, the method allows for robust generation and iterative refinement of the corresponding target molecules, ensuring that the targeted molecule meets the specified properties with high precision by utilizing combination of Variational Autoencoders (VAEs) for encoding and decoding along with evolutionary algorithms. The use of fitness score ensures continuous improvement and prioritizes the corresponding target molecules that best meet the specified criteria.

In another aspect, the present disclosure provides a system for generating a target molecule. The system comprises a processor. The processor is configured to receive a first user input indicative of properties associated with the target molecule and identify a plurality of properties associated with the targeted molecule and a plurality of corresponding objectives, therefrom, wherein each property amongst the plurality of properties is associated with a corresponding objective amongst the plurality of corresponding objectives. Moreover, the processor is configured to generate property scores for the identified plurality of properties using a property predictor algorithm. Furthermore, the processor is configured to receive a second user input indicative of a molecular structure of an input molecule. Furthermore, the processor is configured to generate corresponding target molecules, based on the molecular structure of the input molecule, using a Variational Autoencoder (VAE) module. Furthermore, the processor is configured to generate embeddings of the corresponding target molecules, using a contrastive pretrained molecule encoder. Furthermore, the processor is configured to determine an aggregate similarity score based on similarity scores between the embeddings of the target molecules and embeddings of key relevant information extracted from the first user input. Furthermore, the processor is configured to determine an aggregate property score based on the identified plurality of objectives and the property scores of the identified plurality of properties. Furthermore, the processor is configured to determine fitness scores of the corresponding target molecules, based on the aggregate similarity score and the aggregate property score. Furthermore, the processor is configured to determine whether a given target molecule amongst the corresponding target molecules fulfill a termination criteria and when it is determined that the termination criteria is fulfilled by the given target molecule, deem the given target molecule as the target molecule to be generated, or when it is determined that the termination criteria is not fulfilled by the given target molecule, update the generated corresponding target molecules and iteratively perform steps (v) to (ix).

The system achieves all the advantages and technical effects of the method of the present disclosure.

It has to be noted that all devices, elements, circuitry, units and means described in the present application could be implemented in the software or hardware elements or any kind of combination thereof. All steps which are performed by the various entities described in the present application as well as the functionalities described to be performed by the various entities are intended to mean that the respective entity is adapted to or configured to perform the respective steps and functionalities. Even if, in the following description of specific embodiments, a specific functionality or step to be performed by external entities is not reflected in the description of a specific detailed element of that entity which performs that specific step or functionality, it should be clear for a skilled person that these methods and functionalities can be implemented in respective software or hardware elements, or any kind of combination thereof. It will be appreciated that features of the present disclosure are susceptible to being combined in various combinations without departing from the scope of the present disclosure as defined by the appended claims.

Additional aspects, advantages, features, and objects of the present disclosure would be made apparent from the drawings and the detailed description of the illustrative implementations construed in conjunction with the appended claims that follow.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not too scaled. Wherever possible, like elements have been indicated by identical numbers.

Embodiments of the present disclosure will now be described, by way of example only, with reference to the following diagrams wherein:

FIGS. 1A and 1B collectively are a flowchart of a method for generating a target molecule, in accordance with an embodiment of the present disclosure;

FIG. 2 is a flowchart depicting of an exemplary scenario depicting pre-training steps of VAE, based on the molecular structure of the input molecule, in accordance with an embodiment of the present disclosure;

FIG. 3 is a flowchart depicting of an exemplary scenario depicting steps for generating embeddings of the corresponding target molecules, using a contrastive pretrained molecule encoder, in accordance with an embodiment of the present disclosure;

FIG. 4 is a flowchart depicting of an exemplary scenario depicting steps of genetic algorithm optimization, in accordance with an embodiment of the present disclosure;

FIG. 5 is a flowchart depicting of an exemplary scenario depicting steps of determining whether a given target molecule amongst the corresponding target molecules fulfill a termination criteria, in accordance with an embodiment of the present disclosure;

FIG. 6 is a flowchart depicting of an exemplary scenario depicting steps of filtering process of the corresponding target molecules, in accordance with an embodiment of the present disclosure;

FIG. 7 is a flowchart depicting of an exemplary scenario depicting steps of screening of the corresponding target molecules, in accordance with an embodiment of the present disclosure; and

FIG. 8 is a schematic implementation of a system for generating a target molecule, in accordance with an embodiment of the present disclosure, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF THE DISCLOSURE

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

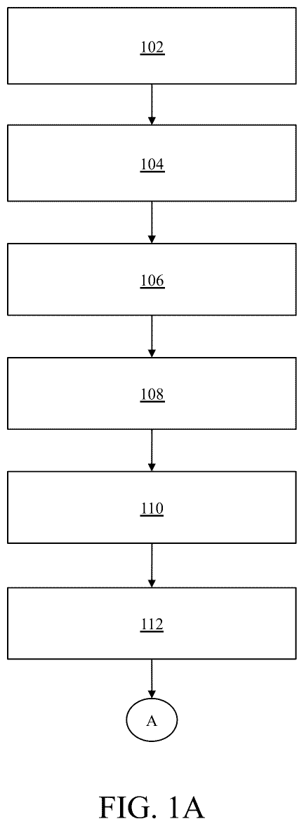

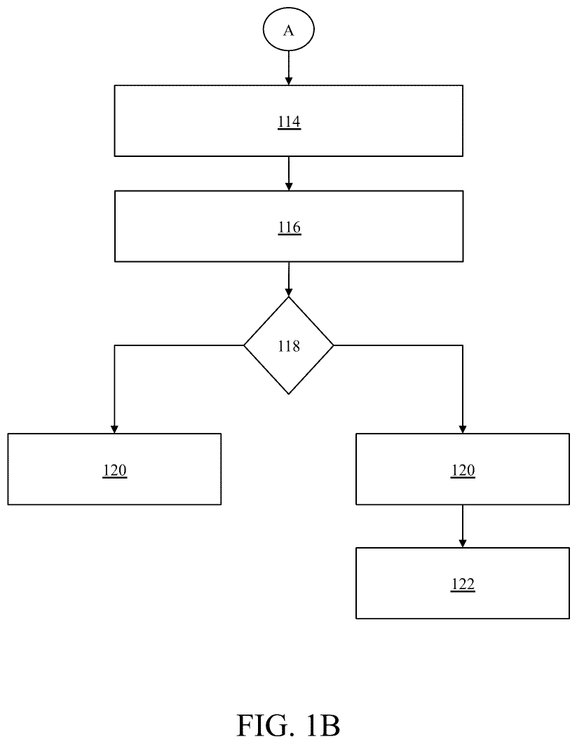

FIGS. 1A and 1B collectively are a flowchart of a method for generating a target molecule, in accordance with an embodiment of the present disclosure. The method 100 comprises steps from 102 to 122.

Herein, the term “target molecule” refers to a specific molecule that is to be generated in the process of drug discovery, due to having desired molecular properties such as drug likeness score, solubility and less toxicity. Typically, the target molecule is highly aligned with user-defined properties and objectives. The target molecule that is generated acts as a potential drug solution obtained in the process of drug discovery. At step 102, a first user input indicative of properties associated with a target molecule is received and pluralities of properties associated with a targeted molecule are identified. Herein, the term “first user input” refers to an input or instruction provided by the user that contains information about desired molecule property constraints and behavior in form of the properties associate with the target molecule. Typically, the first user input can be a document such as a clinical trial report. The properties may include, but are not limited to, physical, chemical, biological or pharmacological attributes. Moreover, the first user input can be provided in various forms such as textual descriptions, numerical values, and the like, that describe the properties associated with the target molecule. Furthermore, the first user input indicative of properties associated with the target molecule allows to capture the user's requirements and preferences for the target molecule. Furthermore, the user interacts with a graphical or textual interface to provide the first input.

It will be appreciated that the first user input is used for extracting key relevant information, using Natural Language Processing (NLP) algorithm. Typically, the purpose of using the natural language processing (NLP) algorithm is to accurately interpret and translate the first user input, which may contain complex and unstructured information about the target molecule properties and objectives. The term “key relevant information” refers to a critical piece of data such as drug information or purpose of the target molecule such as for anti-inflammatory preparations, extracted from the first user input, which is pertinent for generating the target molecule. Herein, the term “natural language processing algorithm” refers to a processing technique that is used to understand, interpret and extract the relevant information from the first user input. Moreover, the NLP algorithm is employed to extract the key relevant information from the first user textual input by performing tasks such as tokenization, part-of-speech tagging, named entity recognition, and semantic analysis.

Notably, embeddings of the extracted key relevant information are generated, using a contrastive pretrained text encoder. Herein, term “contrastive pretrained text encoder” refers to a text encoder that processes the extracted key relevant information from the first user input and generates a vector embedding representation for each key relevant information. Typically, the contrastive pretrained text encoder transforms extracted key relevant information into dense vector representations (embeddings) in a way that similar information has similar embeddings. Notably, the contrastive pretrained text encoder uses contrastive learning techniques that involves training the model to distinguish between similar and dissimilar pairs of text samples. Beneficially, the contrastive pretrained text encoder brings the embeddings of similar text closer together and pushes the embeddings of dissimilar texts further apart in the vector space. Moreover, the relevant information extracted from the first user input, which includes properties and objectives related to the target molecule, is fed into the contrastive pretrained text encoder. The contrastive pretrained text encoder processes the first user input and generates embeddings for each piece of the extracted information. The embeddings capture semantic meaning and contextual relationships within the key relevant information.

Subsequently, the plurality of properties associated with the target molecule and the plurality of corresponding objectives are identified, using a Named Entity Recognition (NER) algorithm. Notably, the plurality of corresponding objectives might specify the desired range or target values for the plurality of properties associated with the target molecules. Throughout the present disclosure, the term “Named Entity Recognition algorithm” refers to a recognition algorithm that is used to identify and classify the properties associated with the target molecule within the first user input, into predefined categories such as molecule binding capacity with a specific target protein, solubility in water, molecular weight and the like. Notably, the Named Entity Recognition (NER) algorithm identifies the plurality of properties such as solubility, toxicity and molecular weight. Each property amongst the plurality of properties identified from the first user input is linked with a corresponding objective such as maximization, minimization, property target. For example, the Quantitative Estimate of Drug-likeness (QED) is mapped to maximization, while PLogP is mapped to a target value of 2.5. Moreover, purpose of the NER algorithm is to ensure clear identification and categorization of the plurality of properties and the plurality of corresponding objectives. Subsequently, the NER algorithm scans the extracted information to locate and classify mentions of the plurality of properties and the plurality of corresponding objectives in the key relevant information. For example, the NER algorithm recognizes terms related to plurality of properties (for example, “solubility”, “toxicity”) in the key relevant information and links each of the plurality of properties with the corresponding objective (for example, “high solubility” means maximizing the solubility) mentioned in the key relevant information.

At step 104, property scores are generated for the identified plurality of properties using a property predictor algorithm. Herein, the term “property predictor algorithm” refers to a predictor algorithm used by a property predictor to generate property scores for the identified plurality of properties of the targeted molecule. Typically, the property predictor algorithm generates the property scores in numerical terms. Moreover, generation of the property scores provides quantitative measures of how well the target molecule meets the desired criteria. The property scores facilitate evaluation and comparison between different molecules based on the identified plurality of properties, guiding the selection and optimization process to ensure that the target molecule possesses the desired properties. Beneficially, the property predictor algorithm automates the process of property evaluation, allowing for rapid assessment of numerous molecules without the need for extensive experimental testing.

In an implementation, the property predictor algorithm is one of: an RD Kit, a deep learning model. Herein, the term “RD Kit” refers to an open-source cheminformatics software library that is used to predict properties of molecules based on the chemical structures. Typically, the RD Kit is able to handle various chemical structure representations, including SMILES (Simplified Molecular Input Line Entry System), InChI (International Chemical Identifier), and molecular graphs. Moreover, the RD Kit takes the molecular structure as input, typically in the form of SMILES or another standard representation and outputs the generated property scores. Beneficially, the RD Kit automates the calculation of the property scores, allowing for high-throughput screening of large chemical libraries. The term “deep learning model” refers to an artificial intelligence (AI) model based on neural networks that is used to predict properties of the molecules and generate the property scores. Typically, the deep learning model consists of multiple layers of neurons, each layer extracting increasingly abstract features from the first user input. Beneficially, the deep learning model is able to identify complex patterns and relationships within large datasets in cheminformatics for predicting molecular properties. Furthermore, the identified plurality of properties associated with the target molecule is provided as input to the deep learning model. The deep learning model is trained on a dataset of molecules with known properties. During the training, the model learns to map the input molecular structures to the corresponding properties and subsequently, provide the property scores as the output. A technical effect is that the RD Kit and the deep learning model is able to achieve high accuracy in generating property scores due to their ability to learn from large datasets.

At step 106, a second user input indicative of a molecular structure of an input molecule is received. The term “input molecule” refers to that specific molecule provided by the user as a starting point for the molecule generation and drug discovery process for generating the target molecule. Typically, the input molecule is directly specified by the user through the second user input. The term “molecular structure” refers to a specific atomic and bonding configuration of the input molecule provided by the user. Typically, the molecular structure serves as an initial template that is modified and adjusted for the molecule generation and drug discovery process. Herein, the term “second user input” refers to an input provided by the user that indicates molecular structure of the input molecule. Typically, the second user input can be in the form of a chemical structure, a molecular formula and the like.

In an implementation, the molecular structure of the input molecule is in form of a SELFIES representation. Herein, the term “SELFIES representation” refers to a novel string-based molecular notation that stands for Self-Referencing Embedded Strings, of the molecular structure of the input molecule. Advantageously, the SELFIES representation of the input molecule reduces errors and increases the efficiency of the algorithm by avoiding the need to validate the chemical structures of the input molecule separately. A technical effect of the SELFIES representation is to avoid computational overhead associated with validating and correcting invalid molecular structure, thereby speeding up the molecular generation process.

At step 108, corresponding target molecules are generated, based on the molecular structure of the input molecule, using a Variational Autoencoder (VAE) module. Herein, the term “Variational Autoencoder (VAE) module” refers to a neural network module designed for unsupervised generation of the corresponding target molecules using the molecular structure of the input molecule. The VAE module is used to learn a compact, continuous latent representation of the input molecule. The latent representation enables efficient exploration and generation of the corresponding target molecules that retain the desired properties of the input molecule. Herein, the term “corresponding target molecules” refers to new target molecules generated as potential candidates to be the target molecule, by the VAE module, using the molecular structure on the input molecule. Typically, the corresponding target molecules are generated to create novel compounds that retain the desired properties of the input molecule while potentially exhibiting improved or tailored characteristics required to be the target molecule having the identified plurality of properties.

In an implementation, the step of generating the corresponding target molecules, based on the molecular structure of the input molecule comprises:
encoding the molecular structure of the input molecule for generating a latent vector representation of the input molecule within a latent space, using a VAE encoder;
initializing a population of candidate latent vectors from the generated latent vector representation within the latent space; and
decoding the population of candidate latent vectors for generating the corresponding target molecules, using a VAE decoder.

In this regard, the term “variational autoencoder encoder” refers to a neural network based encoder that encodes the molecular structure of the input molecule into a latent vector representation. Typically, the variational autoencoder (VAE) encoder compresses the input molecule into a latent space. Herein, the term “latent vector representation” refers to the compressed and abstracted version of the molecular structure of the input molecule represented in the latent space. Typically, the latent vector representation is generated by encoding the input molecule. Beneficially, the latent vector representation captures the essential features and properties of the input molecule and encodes meaningful structural and chemical information about the input molecule in the latent space. The term “latent space” refers to a continuous, multi-dimensional space in which the latent vector representation of the input molecule is continuously represented. Moreover, the latent space allows for interpolation and extrapolation of the input molecule. Furthermore, the VAE encoder takes the molecular structure of the input molecule and maps the molecular structure to the lower-dimensional latent space where each point represents a different molecular configuration. Herein, the term “candidate latent vectors” refers to a set of latent vectors that are generated from the latent vector representation within the latent space to serve as starting points for the generating the corresponding target molecules. Notably, initializing the population of candidate latent vectors involves selecting or creating a diverse set of initial points within the latent space. Each candidate latent vector encapsulates a different configuration or characteristic of the input molecule. Optionally, if no input molecule is provided to the VAE encoder, then the VAE encoder may randomly sample the latent space to create the candidate latent vectors. Moreover, the generated latent vector representation of the input molecule is used for generating the population of candidate latent vectors. The population of candidate latent vectors typically consists of multiple vectors that are strategically chosen or randomly generated to ensure diversity and coverage of the latent space. Each candidate vector represents a potential solution or configuration for further evaluation. Herein, the term “VAE decoder” refers to a neural network-based decoder that converts the population of candidate latent vectors into the molecular structure to generate the corresponding target molecules. Notably, generation of the corresponding target molecules from the latent representations is crucial for evaluating the generated molecules against the plurality of properties and the plurality of corresponding objectives associated with the target molecule. Moreover, the population of the candidate latent vectors, which are points in the latent space, serve as input to the VAE decoder. The VAE decoder processes the candidate latent vectors through a series of transformations such as neural network layers to generate the molecular structure in the format such as the SELFIES representation. The VAE decoder generates diverse molecules that are consistent with the candidate latent vectors, capturing the chemical and structural diversity of the training data, and identification of novel molecules with desired properties for generating the corresponding target molecules. A technical effect is that the corresponding target molecules are effectively and accurately generated using a well-defined and reliable process.

In an implementation, subsequent to the step of initializing the population of candidate latent vectors from the generated latent vector representation within the latent space, the step of generating the corresponding target molecules, based on the molecular of the input molecule further comprises:
mutating the population of the candidate latent vectors for diversifying the population of the candidate latent vectors, using a differential mutation operator; and
combining the mutated population of the candidate latent vectors for updating the generated corresponding target molecules.

In this regard, the term “differential mutation operator” refers to a mutation operator within the Differential Evolution (DE) algorithm that creates a mutant vector by adding weighted difference between two randomly chosen candidate vectors to a third candidate vector. Moreover, by applying the differential mutation operation, small changes to the latent vector representations of the population of the candidate latent vectors are performed and the population of the candidate latent vectors is diversified. Beneficially, diversification in the population of the candidate latent vectors facilitates preventing premature convergence to suboptimal solutions and enhances more guided exploration of the latent space. The differential mutation operator selects, for example, three distinct vectors x1, x2 and x3 randomly from the current population of the candidate latent vectors and calculates difference between the two latent vectors (x2-x3). Subsequently, the difference is scaled by a factor F (a parameter that controls the extent of mutation). Then adding the scaled difference to the third vector (x1) to create a new mutant vector (v=x1+F.(x2-x3)). Notably, combining the mutated population of the candidate latent vectors incorporates the diversity and ensures that the best candidate latent vectors are retained, and the overall quality of the generated corresponding target molecules improves. A technical effect is that iterative process of mutation and updating the generated corresponding target molecules allows the algorithm to adapt to new information, making the search process more dynamic and robust. Furthermore, the aforementioned process maintains a balance between exploring new areas (through mutation) and exploiting known good solutions (through combining and updating), leading to a more efficient and comprehensive search for optimal solutions.

At step 110, embeddings of the corresponding target molecules are generated using a contrastive pretrained molecule encoder. Herein, the term “contrastive pretrained molecule encoder” refers to a molecule encoder that processes the target molecules and generates a vector embedding representation for the corresponding target molecule. Typically, the contrastive pretrained molecule encoder transforms information of the corresponding target molecules into dense vector representations (embeddings). Beneficially, the contrastive pretrained molecule encoder brings the embeddings of similar corresponding target molecules closer together and pushes the embeddings of dissimilar corresponding target molecules further apart in the vector space. Moreover, the relevant information extracted from the corresponding target molecules, which includes properties and objectives related to the corresponding target molecules, is fed into the contrastive pretrained molecule encoder. The embeddings capture semantic meaning and contextual relationships within the corresponding target molecules. The use of contrastive pretraining ensures that the embeddings are robust and capture subtle differences and similarities between the target molecules.

At step 112, an aggregate similarity score is determined based on similarity scores between the embeddings of the corresponding target molecules and embeddings of key relevant information extracted from the first user input. Herein, the term “aggregate similarity score” refers to a numerical value that represents the aggregation of degree of cosine similarity between the embeddings of the target molecules and the embeddings of key relevant information extracted from the first user input. Typically, the aggregate similarity score is determined to ensure that the corresponding generated target molecules align closely with the plurality of properties and the plurality of corresponding objectives extracted from the first user input. Moreover, the aggregate similarity score provides feedback on how well the generated corresponding target molecules align the plurality of properties and the plurality of corresponding objectives, where the aggregate similarity score having a higher value indicate better alignment of the generated corresponding target molecules with the plurality of properties and the plurality of corresponding objectives. The embeddings of the target molecules and the extracted key relevant information are compared pairwise. For example, cosine similarity measures the cosine of the angle between two vectors, providing a value between −1 and 1, where 1 indicates identical direction (maximum similarity), and −1indicates opposite direction (minimum similarity). Once individual similarity scores are calculated for each pair of embeddings, then the similarity scores are aggregated (e.g., averaged) to produce the aggregate similarity score that represents the overall alignment between the embeddings of the generated corresponding target molecules and the plurality of properties and the plurality of corresponding objectives.

At step 114, an aggregate property score is determined based on the identified plurality of objectives and the property scores of the identified plurality of properties. Herein, the term “aggregate property score” refers to a numerical value of the aggregation of property scores that represents how well the plurality of properties of the target molecule meets the identified plurality of objectives. Moreover, determining the aggregate property score is used to quantitatively assess and compare how well the generated target molecules meet the plurality of corresponding objectives of the plurality of properties. Notably, the molecule property predictor is utilized to compute the property scores. Furthermore, the property scores are determined by comparing the actual value of a property of the target molecule against a predefined objective or target value. The comparison can be made using various methods, such as difference, ratio, or more complex scoring functions. For each property, a specific scoring function is defined that translates the difference between the actual property value and the target value into a score. For instance, if a given property has the corresponding objective of maximization, the property score might be higher if the property value is closer to or exceeds a specific value. Once individual property score are calculated for each property, then the individual property scores for the plurality of properties are aligned with the plurality of corresponding objectives to determine the aggregate property score that reflects the target molecule's overall fitness relative to the plurality of corresponding objectives.

At step 116, fitness scores of the corresponding target molecules are determined, based on the aggregate similarity score and the aggregate property score. Herein, the term “fitness scores” refers to a numerical value that indicates how fit or suitable each of the corresponding target molecules to be the target molecule. Typically, determining the fitness scores is essential for evaluating and ranking the corresponding target molecules in terms of overall suitability for a given application. The fitness scores are determined based on the aggregate similarity scores and the aggregate property scores through various mathematical methods such as weighted sums, averages, and the like. Furthermore, the fitness scores are used to rank the corresponding target molecules and select those corresponding target molecules with high fitness scores are selected as parent molecules based on the fitness scores. The corresponding target molecules with higher fitness scores are considered more suitable to select as the parent molecules as those corresponding target molecules have both higher similarity score and higher property score. Furthermore, in cases where the corresponding target molecules have identical fitness scores, additional criteria (e.g., specific property scores, novelty, or computational efficiency) can be used to adjust the fitness scores.

In an implementation, subsequent to step 116 and prior to step 118, the method further comprises:
filtering the corresponding target molecules using a toxicity filter; identifying a second set of target molecules amongst the corresponding target molecules that fails to pass the toxicity filter; and
removing the second set of target molecules amongst the corresponding target molecules.

In this regard, the term “toxicity filter” refers to a filter that is used to identify and exclude the corresponding target molecules that exhibit potential toxic effects based on the fitness scores of the corresponding target molecules. Typically, the toxicity filter assesses the molecular structures and predicts the likelihood of causing adverse biological effects, thereby ensuring that only non-toxic or minimally toxic corresponding target molecules pass from the filtering process. The term “second set of target molecules” refers to those harmful target molecules that do not meet the toxicity criteria and have low fitness scores and fail to pass the toxicity filter. Notably, the second set of target molecules posses'toxic properties which can cause adverse effects. Moreover, the toxicity filter uses toxicity classifier to identify the second set of target molecules. Furthermore, the evolutionary algorithm employs the toxicity filter to remove the second set of molecules amongst the corresponding target molecules. A technical effect is that by removing the toxic corresponding target molecules from the corresponding target molecules, the method reduces the risk of harmful candidates being identified as the target molecule.

In an implementation, subsequent to step 116 and prior to step 118, the method further comprises:
screening the corresponding target molecules for identifying a third set of target molecules amongst the corresponding target molecules having a binding affinity lower than a threshold value; and
removing the third set of target molecules amongst the corresponding target molecules.

In this regard, the term “binding affinity” refers to a strength of interaction between the corresponding target molecules and its target such as a protein, enzyme or receptor. Typically, the binding affinity measures how tightly the corresponding target molecules bind to the target. The term “threshold value” refers to a pre-defined value that sets the minimum acceptable binding affinity for the corresponding target molecules to be considered effective. The term “third set of target molecules” refers to those corresponding target molecules identified during the screening process that has lower binding affinity than the threshold value of the corresponding target molecules. Moreover, the purpose of the screening is to identify the third set of target molecules amongst the corresponding target molecules with insufficient binding affinity to be removed. Furthermore, the binding affinity of each corresponding target molecule amongst the corresponding target molecules is determined using deep learning algorithms and computational methods (such as genetic algorithm and differential evolution), experimental assays, or a combination thereof. The calculated binding affinity of each target molecule is compared to the threshold value. The target molecules with binding affinity values below the threshold value are identified and classified into the third set of target molecules to be removed amongst the corresponding target molecules. A technical effect is that by removing the target molecules with lower binding affinity than the threshold value, the overall quality of the corresponding target molecules based on the binding affinity is improved.

In step 118, whether a given target molecule amongst the corresponding target molecules fulfill a termination criteria is determined. Herein, the term “termination criteria” refers to a specific set of criteria such as binding affinity, toxicity level, molecular stability and the like that signify that the given target molecule is suitable to be deemed as the target molecule to be generated. The term “given target molecule” refers to a randomly selected target molecule amongst the corresponding target molecules that is evaluated to determine whether the termination criteria is met or not by the given target molecule. Moreover, the given target molecule and the corresponding target molecules are assessed against the termination criteria using computational models, experimental data and the like to determine whether the given target molecule and the corresponding target molecules fulfil the termination criteria.

When it is determined that the termination criteria is fulfilled by the given target molecule, at step 120, the given target molecule is deemed as the target molecule to be generated. Herein, the target molecule is successfully generated in form of the given target molecule that successfully fulfills the termination criteria. Furthermore, deeming the given target molecule as the target molecule acts as an end point for the drug discovery process, saving computational resources and time.

Alternatively, when it is determined that the termination criteria is not fulfilled by the given target molecule, at step 120, the generated corresponding target molecules are updated. Herein, once it is determined that the given target molecule is not able to fulfill the termination criteria, then the population of the candidate latent vectors are updated with the offspring of the parent molecules. Typically, updating the population of the candidate latent vectors ensures that new target molecules in form of the offspring molecules are added to the corresponding target molecules to ensure that the given target molecule that fulfills the termination criteria is found. Moreover, the method uses techniques like mutation, crossover and selection to generate the new set of candidate latent vectors.

Moreover, when it is determined that the termination criteria is not fulfilled by the given target molecule, at step 122, steps 110 to 118 are performed iteratively. It will be appreciated that the iterative process ensures continuous modification and updating of the corresponding target molecules till the given target molecule amongst the corresponding target molecules that fulfills the termination criteria is found. By repeatedly performing the steps 110 to 118, the method refines the corresponding target molecules, aligning them more closely with the plurality of properties and the plurality of corresponding objectives associated with the target molecule in order to increase the probability of finding the given target molecule amongst the corresponding target molecules that fulfills the termination criteria.

In an implementation, when it is determined that the termination criteria is not fulfilled by the given target molecule, subsequent to step 118 and prior to step 120, the method further comprises:
identifying a first set of target molecules amongst the corresponding target molecules as parent molecules, based on the fitness scores of the corresponding target molecules;
generating latent vectors of the parent molecules, using the VAE encoder; combining latent vectors of the parent molecules for generating latent vectors of offspring molecules;
mutating the latent vectors of the offspring molecules for diversifying the latent vectors of the offspring molecules, using a differential mutation operator; and
using the mutated latent vectors of the offspring molecules for updating the population of the candidate latent vectors.

In this regard, the term “first set of target molecules” refers to a subset of the target molecules that is selected from the corresponding target molecules, having high fitness scores which makes them suitable to be selected as the parent molecules. Notably, the first set of target molecules used as parent molecules for producing the offspring molecules. Herein, the term “parent molecules” refers to the first set of molecules that will be used to generate new target molecules (i.e., the offspring molecules). Typically, those corresponding target molecules having the highest fitness scores, are selected as the parent molecules. Beneficially, selecting those corresponding target molecules having the highest fitness scores as the parent molecules leads to the generation of the offspring molecules with potentially better properties.

Moreover, the latent vectors of the parent molecules are generated using the VAE encoder. The latent vectors provide a manageable and structured representation of the parent molecules, and makes easier to perform genetic operations such as crossover, in the latent space. Furthermore, the latent vectors of the offspring molecules are generated by combining the latent representations of the parent molecules with potentially improved properties. Typically, the latent vectors of the parent molecules are combined using a crossover operation. Moreover, the crossover operation can be single-point crossover, multi-point crossover, arithmetic crossover and the like. Furthermore, the result of the crossover operation is a new set of latent vectors that represent the offspring molecules. Beneficially, combining the latent vectors of the parent molecules introduces genetic diversity in the generated corresponding target molecules.

Furthermore, the term “differential mutation operator” refers to a mutation operator that introduces small, random biological changes to the latent vectors of the offspring molecules. Typically, by mutating the latent vectors of the offspring molecules, genetic diversity of the offspring molecules can be increased. Furthermore, the differential mutation operator randomly selects three latent vectors of the parent molecules and calculates the difference between two of the selected latent vectors. Subsequently, the differential mutation operator multiplies the difference by a scaling factor (mutation factor) to control the magnitude of the mutation and adds the scaled difference to the third latent vector to generate a new mutated vector of the parent molecules. The resulting vector is the mutated offspring vector that has diverse properties compared to the parent molecules. Furthermore, the mutated latent vectors of the offspring molecules are integrated back into the population of the candidate latent vectors. The purpose is to update the population of the candidate latent vectors with the newly created diversified mutated latent vectors of the offspring molecules. A technical effect is that the population of the candidate latent vectors is effectively updated and diversified for generating diversified corresponding target molecules.

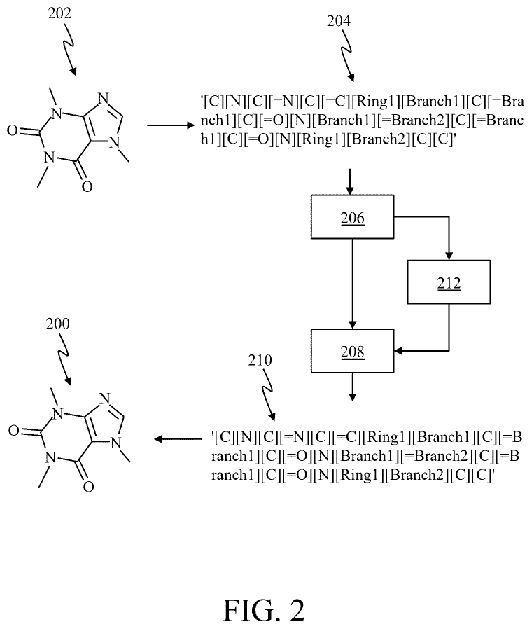

FIG. 2 is a flowchart of an exemplary scenario depicting pre-training steps of VAE, based on a molecular structure 202 of an input molecule 204, in accordance with an embodiment of the present disclosure. At step 206, the molecular structure 202 of the input molecule 204 is encoded for generating a latent vector representation (Z) of the input molecule 204 within a latent space, using a VAE encoder. At step 208, the population of candidate latent vectors is decoded for generating the corresponding target molecules 200 depicted as corresponding molecular structures (depicted as a first corresponding molecular structure 210) thereof, using a VAE decoder. Optionally, at step 212, a population of candidate latent vectors is initialized from the generated latent vector representation within the latent space.

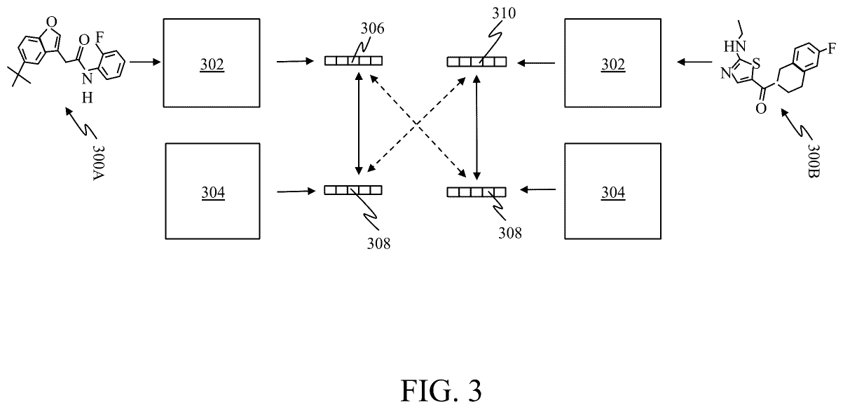

FIG. 3 is a flowchart depicting of an exemplary scenario depicting steps for generating embeddings of the corresponding target molecules, using a contrastive pretrained molecule encoder, in accordance with an embodiment of the present disclosure. At step 302A, the contrastive pretrained molecule encoder receives the VAE-decoded corresponding target molecules. At step 302B, a contrastive pretrained text encoder receives the extracted drug constraint information from the first user input. At step 304, the contrastive pretrained molecule encoder maps the molecule and the text into an embedding space. At step 306, projector module aligns the embeddings of positive molecule-text pairs and differentiate those of negative pairs.

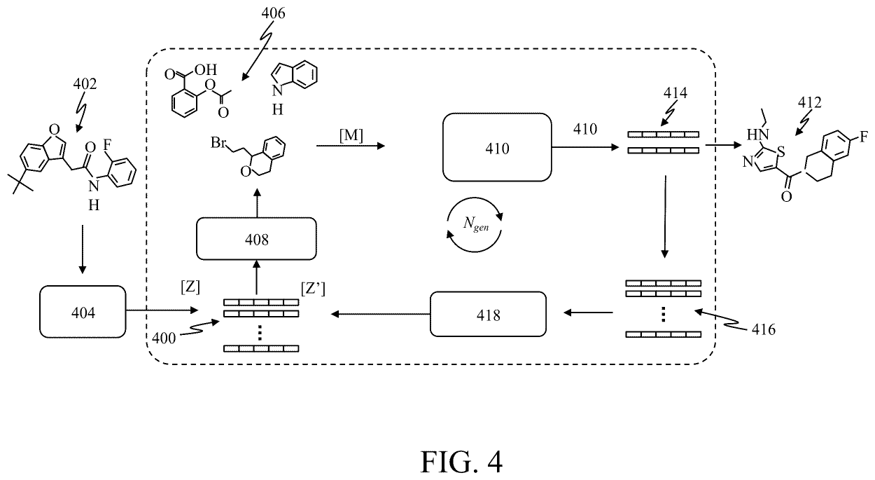

FIG. 4 is a schematic illustration depicting of an exemplary scenario depicting steps of updating a population of candidate vectors 400, in accordance with an embodiment of the present disclosure. As shown, a molecular structure 402 of an input molecule is encoded for generating a latent vector representation of the input molecule within a latent space, using a VAE encoder 404. Subsequently, a population of candidate latent vectors 400 are initialized from the generated latent vector representation within the latent space. Subsequently, the population of candidate latent vectors 400 are decoded for generating the corresponding target molecules 406, using a VAE decoder 408. Subsequently, an aggregate property score is determined based on identified plurality of objectives and property scores of identified plurality of properties, and fitness scores 410 of the corresponding target molecules 406 are determined based on the aggregate similarity score and the aggregate property score. Subsequently, it is determined whether a given target molecule amongst the corresponding target molecules 406 fulfill a termination criteria. Subsequently, when it is determined that the termination criteria is fulfilled by the given target molecule, the given target molecule is deemed as a target molecule 412 to be generated. Alternatively, when it is determined that the termination criteria is not fulfilled by the given target molecule, a first set of target molecules are identified amongst the corresponding target molecules 406 as parent molecules 414 based on the fitness scores 410 of the corresponding target molecules 406. Subsequently, latent vectors of the parent molecules 414 are combined for generating latent vectors of offspring molecules 416. Subsequently, the latent vectors of the offspring molecules 416 are mutated for diversifying the latent vectors of the offspring molecules using a differential mutation operator 418. Subsequently, the mutated latent vectors of the offspring molecules are used for updating the population of the candidate latent vectors 400.

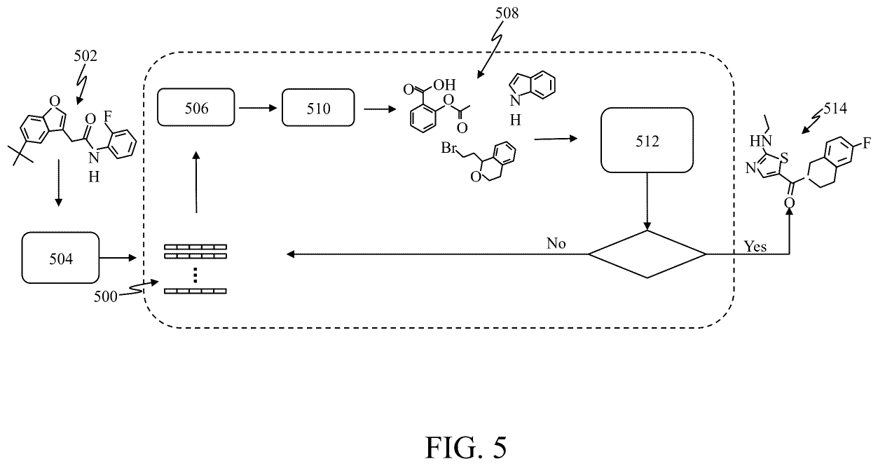

FIG. 5 is a schematic illustration depicting of an exemplary scenario depicting steps of updating a population of candidate vectors 500, in accordance with another embodiment of the present disclosure. As shown, a molecular structure 502 of an input molecule is encoded for generating a latent vector representation of the input molecule within a latent space, using a VAE encoder 504. Subsequently, a population of candidate latent vectors 500 are initialized from the generated latent vector representation within the latent space. Subsequently, the population of the candidate latent vectors 500 are mutated for diversifying the population of the candidate latent vectors 500, using a differential mutation operator 506 for updating the mutated population of the candidate latent vectors. Subsequently, the updated population of the candidate latent vectors are decoded for generating updated corresponding target molecules 508, using a VAE decoder 510. Subsequently, an aggregate property score is determined based on identified plurality of objectives and property scores of identified plurality of properties, and fitness scores 512 of the corresponding target molecules 508 are determined based on the aggregate similarity score and the aggregate property score. Subsequently, it is determined whether a given target molecule amongst the corresponding target molecules 508 fulfills a termination criteria. Subsequently, when it is determined that the termination criteria is fulfilled by the given target molecule, the given target molecule is deemed as a target molecule 514 to be generated. Alternatively, when it is determined that the termination criteria is not fulfilled by the given target molecule, the corresponding target molecules 508 are updated.

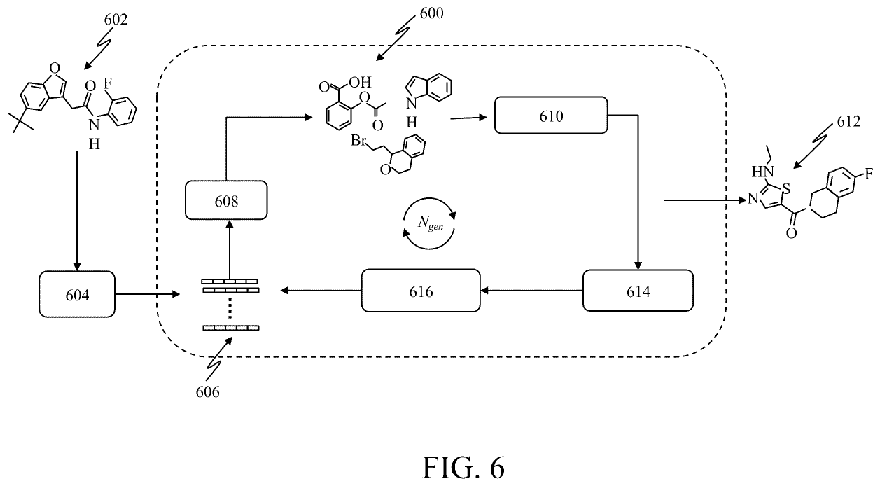

FIG. 6 is a schematic illustration depicting of an exemplary scenario depicting steps of filtering corresponding target molecules 600, in accordance with an embodiment of the present disclosure. As shown, a molecular structure 602 of an input molecule is encoded for generating a latent vector representation of the input molecule within a latent space, using a VAE encoder 604. Subsequently, a population of candidate latent vectors 606 are initialized from the generated latent vector representation within the latent space. Subsequently, the population of candidate latent vectors 606 are decoded for generating the corresponding target molecules 600, using a VAE decoder 608. Subsequently, the corresponding target molecules 600 are filtered using a toxicity filter 610. Subsequently, a second set of target molecules 612 are identified amongst the corresponding target molecules 600 that fails to pass the toxicity filter 610. Subsequently, the second set of target molecules 612 are removed amongst the corresponding target molecules 600. Subsequently, a first set of target molecules are identified amongst remaining corresponding target molecules as parent molecules 614 based on fitness scores of the remaining corresponding target molecules. Subsequently, latent vectors of the parent molecules 614 are combined for generating latent vectors of offspring molecules. Subsequently, the latent vectors of the offspring molecules are mutated for diversifying the latent vectors of the offspring molecules using a differential mutation operator 616. Subsequently, the mutated latent vectors of the offspring molecules are used for updating the population of the candidate latent vectors 606.

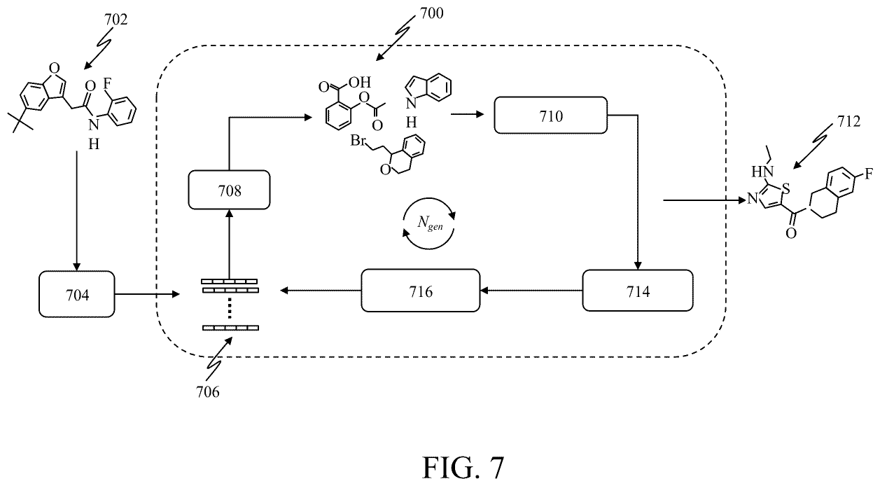

FIG. 7 is a schematic depicting of an exemplary scenario depicting steps of screening of corresponding target molecules 700, in accordance with an embodiment of the present disclosure. As shown, a molecular structure 702 of an input molecule is encoded for generating a latent vector representation of the input molecule within a latent space, using a VAE encoder 704. Subsequently, a population of candidate latent vectors 706 are initialized from the generated latent vector representation within the latent space. Subsequently, the population of candidate latent vectors 706 are decoded for generating the corresponding target molecules 700, using a VAE decoder 708. Subsequently, the corresponding target molecules 700 are screened using virtual screening 710 for identifying a third set of target molecules 712 are identified amongst the corresponding target molecules 700 having a binding affinity lower than a threshold value. Subsequently, the third set of target molecules 712 are removed amongst the corresponding target molecules 700. Subsequently, a first set of target molecules are identified amongst remaining corresponding target molecules as parent molecules 714 based on fitness scores of the remaining corresponding target molecules. Subsequently, latent vectors of the parent molecules 714 are combined for generating latent vectors of offspring molecules. Subsequently, the latent vectors of the offspring molecules are mutated for diversifying the latent vectors of the offspring molecules using a differential mutation operator 716. Subsequently, the mutated latent vectors of the offspring molecules are used for updating the population of the candidate latent vectors 706.

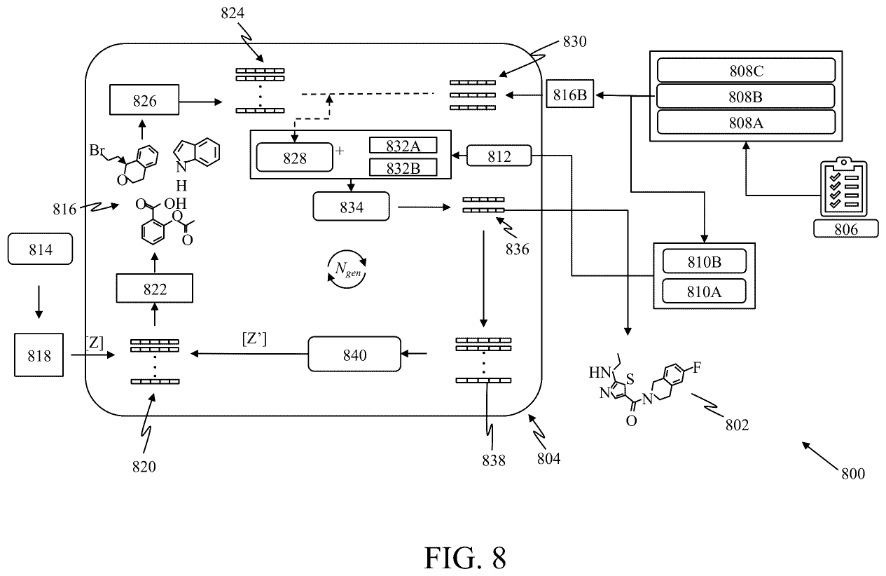

FIG. 8 is a schematic implementation of a system 800 for generating a target molecule 802, in accordance with an embodiment of the present disclosure. As shown, the system 800 comprises a processor 804. The processor 804 is configured to receive a first user input 806 indicative of properties associated with the target molecule 802 and identify a plurality of properties 808A-C associated with the targeted molecule 802 and a plurality of corresponding objectives 810A-B, therefrom, wherein each property amongst the plurality of properties 808A-C is associated with a corresponding objective amongst the plurality of corresponding objectives 810A-B. Moreover, the processor 804 is configured to generate property scores for the identified plurality of properties 808A-C using a property predictor 812 algorithm. Furthermore, the processor 804 is configured to receive a second user input indicative of a molecular structure 814 of an input molecule. Furthermore, the processor 804 is configured to generate corresponding target molecules 816, based on the molecular structure of the input molecule 810, using a Variational Autoencoder (VAE) module. Optionally, to generate the corresponding target molecules 816, based on the molecular of the input molecule 810, the at least one processor 804 is further configured to encode the molecular structure of the input molecule 810 for generating a latent vector representation of the input molecule 810 within a latent space using a VAE encoder 818, initialize a population of candidate latent vectors 820 from the generated latent vector representation within the latent space, and decode the population of candidate latent vectors 820 for generating the corresponding target molecules 816, using a VAE decoder 822. Furthermore, the processor 804 is configured to generate embeddings 824 of the corresponding target molecules 816, using a contrastive pretrained molecule encoder 826. Furthermore, the processor 804 is configured to determine an aggregate similarity score 828 based on similarity scores between the embeddings 824 of the corresponding target molecules 816 and embeddings 830 of key relevant information extracted from the first user input 806. Furthermore, the processor 804 is configured to determine an aggregate property score based on the identified plurality of objectives 810A-B and the property scores 832A-B of the identified plurality of properties 808A-C. Furthermore, the processor 804 is configured to determine fitness scores 834 of the corresponding target molecules 816, based on the aggregate similarity score 828 and the aggregate property score. Furthermore, the at least one processor 804 is configured to determine whether a given target molecule amongst the corresponding target molecules 816 fulfill a termination criteria. Furthermore, when it is determined that the termination criteria is fulfilled by the given target molecule, the at least one processor 804 is configured to deem the given target molecule as the target molecule 802 to be generated. Alternatively, when it is determined that the termination criteria is not fulfilled by the given target molecule, the at least one processor 804 is configured to update the generated corresponding target molecules 816 and iteratively perform the aforementioned steps. Optionally, when it is determined that the termination criteria is not fulfilled by the given target molecule, the at least one processor 804 is configured to identify a first set of target molecules amongst the corresponding target molecules as parent molecules 836, based on the fitness scores 834 of the corresponding target molecules 816, generate latent vectors of the parent molecules 836, using the VAE encoder 818; combine latent vectors (crossover operation) of the parent molecules 836 to generate latent vectors of offspring molecules 838 and mutate the latent vectors of the offspring molecules to diversify the latent vectors of the offspring molecules, using a differential mutation operator 840, and use the mutated latent vectors of the offspring molecules to update the population of the candidate latent vectors 820.

Herein, the term processor 804 refers to a computational element that is operable to execute the software framework. Examples of the processor 804 include, but are not limited to, a microprocessor, a microcontroller, a complex instruction set computing (CISC) microprocessor, a reduced instruction set (RISC) microprocessor, a very long instruction word (VLIW) microprocessor, or any other type of processing circuit. Furthermore, the processor 804 may refer to one or more individual processors, processing devices and various elements associated with a processing device that may be shared by other processing devices. Additionally, one or more individual processors, processing devices and elements are arranged in various architectures for responding to and processing the instructions that execute the software framework.

Modifications to embodiments of the present disclosure described in the foregoing are possible without departing from the scope of the present disclosure as defined by the accompanying claims. Expressions such as “including”, “comprising”, “incorporating”, “have”, “is” used to describe, and claim the present disclosure are intended to be construed in a non-exclusive manner, namely allowing for items, components or elements not explicitly described also to be present. Reference to the singular is also to be construed to relate to the plural. The word “exemplary” is used herein to mean “serving as an example, instance or illustration”. Any embodiment described as “exemplary” is not necessarily to be construed as preferred or advantageous over other embodiments and/or to exclude the incorporation of features from other embodiments. The word “optionally” is used herein to mean “is provided in some embodiments and not provided in other embodiments”. It is appreciated that certain features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable combination or as suitable in any other described embodiment of the disclosure.

What is claimed is:

1. A method for generating a target molecule (412, 514, 802), comprising:
(i) receiving a first user input (806) indicative of properties associated with the target molecule, and identifying a plurality of properties (808A-C) associated with the targeted molecule and a plurality of corresponding objectives (810A-B), therefrom, wherein each property amongst the plurality of properties is associated with a corresponding objective amongst the plurality of corresponding objectives;
(ii) generating property scores (832A-B) for the identified plurality of properties using a property predictor algorithm (812);
(iii) receiving a second user input indicative of a molecular structure (202, 402, 502, 602, 702, 814) of an input molecule (204);
(iv) generating corresponding target molecules (200, 406, 508, 600, 700, 816), based on the molecular structure of the input molecule, using a Variational Autoencoder (VAE) module;
(v) generating embeddings (824) of the corresponding target molecules, using a contrastive pretrained molecule encoder (826);
(vi) determining an aggregate similarity score (828) based on similarity scores between the embeddings of the corresponding target molecules and embeddings (830) of key relevant information extracted from the first user input;
(vii) determining an aggregate property score based on the identified plurality of objectives and the property scores of the identified plurality of properties;
(viii) determining fitness scores (410, 512, 834) of the corresponding target molecules, based on the aggregate similarity score and the aggregate property score;
(ix) determining whether a given target molecule amongst the corresponding target molecules fulfill a termination criteria; and
when it is determined that the termination criteria is fulfilled by the given target molecule:
(x) deeming the given target molecule as the target molecule to be generated; or
when it is determined that the termination criteria is not fulfilled by the given target molecule:
(x) updating the generated corresponding target molecules,
(xi) iteratively performing steps (v) to (ix).

2. The method of claim 1, wherein the step of generating the corresponding target molecules (200, 406, 508, 600, 700, 816), based on the molecular structure (202, 402, 502, 602, 702, 814) of the input molecule (204) comprises:
encoding the molecular structure of the input molecule for generating a latent vector representation of the input molecule within a latent space, using a VAE encoder (404, 504, 604, 704, 818);
initializing a population of candidate latent vectors (400, 500, 606, 706, 820) from the generated latent vector representation within the latent space; and
decoding the population of candidate latent vectors for generating the corresponding target molecules, using a VAE decoder (408, 510, 608, 708, 822).

3. The method according to claim 1, wherein when it is determined that the termination criteria is not fulfilled by the given target molecule, subsequent to step (ix) and prior to step (x), the method further comprises:
identifying a first set of target molecules amongst the corresponding target molecules (200, 406, 508, 600, 700, 816) as parent molecules (414, 614, 714, 836), based on the fitness scores (410, 512, 834) of the corresponding target molecules;
generating latent vectors of the parent molecules, using the VAE encoder (404, 504, 604, 704, 818);
combining latent vectors of the parent molecules for generating latent vectors of offspring molecules (416, 838);
mutating the latent vectors of the offspring molecules for diversifying the latent vectors of the offspring molecules, using a differential mutation operator (418, 506, 616, 716, 840); and
using the mutated latent vectors of the offspring molecules for updating the population of the candidate latent vectors (400, 500, 606, 706, 820).

4. The method according to claim 2, wherein subsequent to the step of initializing the population of candidate latent vectors (400, 500, 606, 706, 820) from the generated latent vector representation within the latent space, the step of generating the corresponding target molecules (200, 406, 508, 600, 700, 816), based on the molecular structure (202, 402, 502, 602, 702, 814) of the input molecule (204) further comprises:
mutating the population of the candidate latent vectors for diversifying the population of the candidate latent vectors, using a differential mutation operator; and
combining the mutated population of the candidate latent vectors for updating the generated corresponding target molecules.

5. The method according to claim 1, wherein subsequent to step (viii) and prior to step (ix), the method further comprises:
filtering the corresponding target molecules (200, 406, 508, 600, 700, 816) using a toxicity filter (610);
identifying a second set of target molecules (612) amongst the corresponding target molecules that fails to pass the toxicity filter; and
removing the second set of target molecules amongst the corresponding target molecules.

6. The method according to claim 1, wherein subsequent to step (viii) and prior to step (ix), the method further comprises:
screening the corresponding target molecules (200, 406, 508, 600, 700, 816) for identifying a third set of target molecules (712) amongst the corresponding target molecules having a binding affinity lower than a threshold value; and
removing the third set of target molecules amongst the corresponding target molecules.

7. The method according to claim 1, wherein the property predictor algorithm (812) is one of: an RD Kit, a deep learning model.

8. The method according to claim 1, wherein the molecular structure (202, 402, 502, 602, 702, 814) of the input molecule (204) is in form of a SELFIES representation.

9. A system (800) for generating a target molecule (412, 514, 802), comprising at least one processor (804) configured to:
(i) receive a first user input (806) indicative of properties associated with the target molecule, and identify a plurality of properties (808A-C) associated with the targeted molecule and a plurality of corresponding objectives (810A-B), therefrom, wherein each property amongst the plurality of properties is associated with a corresponding objective amongst the plurality of corresponding objectives;
(ii) generate property scores (832A-B) for the identified plurality of properties using a property predictor algorithm (812);
(iii) receive a second user input indicative of a molecular structure (202, 402, 502, 602, 702, 814) of an input molecule (204);
(iv) generate corresponding target molecules (200, 406, 508, 600, 700, 816), based on the molecular structure of the input molecule, using a Variational Autoencoder (VAE) module;
(v) generate embeddings (824) of the corresponding target molecules, using a contrastive pretrained molecule encoder (826);
(vi) determine an aggregate similarity score (828) based on similarity scores between the embeddings of the target molecules and embeddings (830) of key relevant information extracted from the first user input;
(vii) determine an aggregate property score based on the identified plurality of objectives and the property scores of the identified plurality of properties;
(viii) determine fitness scores (410, 512, 834) of the corresponding target molecules, based on the aggregate similarity score and the aggregate property score;
(ix) determine whether a given target molecule amongst the corresponding target molecules fulfill a termination criteria; and
when it is determined that the termination criteria is fulfilled by the given target molecule:
(x) deem the given target molecule as the target molecule to be generated; or
when it is determined that the termination criteria is not fulfilled by the given target molecule:
(x) update the generated corresponding target molecules,
(xi) iteratively perform steps (v) to (ix).

10. The system (800) of claim 9, wherein to generate the corresponding target molecules (200, 406, 508, 600, 700, 816), based on the molecular structure (202, 402, 502, 602, 702, 814) of the input molecule (204), the at least one processor (804) is further configured to:
encode the molecular structure of the input molecule for generating a latent vector representation of the input molecule within a latent space, using a VAE encoder (404, 504, 604, 704, 818);
initialize a population of candidate latent vectors (400, 500, 606, 706, 820) from the generated latent vector representation within the latent space; and
decode the population of candidate latent vectors for generating the corresponding target molecules, using a VAE decoder (408, 510, 608, 708, 822).

11. The system (800) according to claim 9, wherein when it is determined that the termination criteria is not fulfilled by the given target molecule, subsequent to step (ix) and prior to step (x), the at least one processor (804) is further configured to:
identify a first set of target molecules amongst the corresponding target molecules (200, 406, 508, 600, 700, 816) as parent molecules (414, 614, 714, 836), based on the fitness scores (410, 512, 834) of the corresponding target molecules;
generate latent vectors of the parent molecules, using the VAE encoder (404, 504, 604, 704, 818);
combine latent vectors of the parent molecules to generate latent vectors of offspring molecules (416, 838);
mutate the latent vectors of the offspring molecules to diversify the latent vectors of the offspring molecules, using a differential mutation operator (418, 506, 616, 716, 840); and
use the mutated latent vectors of the offspring molecules to update the population of the candidate latent vectors (400, 500, 606, 706, 820).

12. The system (800) according to claim 10, wherein subsequent to the step of initializing the population of candidate latent vectors (400, 500, 606, 706, 820) from the generated latent vector representation within the latent space, to generate the corresponding target molecules (200, 406, 508, 600, 700, 816), based on the molecular structure (202, 402, 502, 602, 702, 814) of the input molecule (204), the at least one processor (804) is further configured to:
mutate the population of the candidate latent vectors to diversify the population of the candidate latent vectors, using a differential mutation operator; and
combine the mutated population of the candidate latent vectors to update the generated corresponding target molecules.

13. The system (800) according to claim 9, wherein subsequent to step (viii) and prior to step (ix), the at least one processor (804) is further configured to:
filter the corresponding target molecules (200, 406, 508, 600, 700, 816) using a toxicity filter;
identify a second set of target molecules amongst the corresponding target molecules that fails to pass the toxicity filter; and
remove the second set of target molecules amongst the corresponding target molecules.

14. The system (800) according to claim 9, wherein subsequent to step (viii) and prior to step (ix), the at least one processor (804) is further configured to:
screen the corresponding target molecules (200, 406, 508, 600, 700, 816) to identify a third set of target molecules amongst the corresponding target molecules having a binding affinity lower than a threshold value; and
remove the third set of target molecules amongst the corresponding target molecules.

15. The system (800) according to claim 9, wherein the property predictor algorithm (812) is one of: an RD Kit, a deep learning model.

16. The system (800) according to claim 9, wherein the molecular structure (202, 402, 502, 602, 702, 814) of the input molecule (204) is in form of a SELFIES representation.
